(19) United States
(12) Patent Application Publication
(10) Pub. No.: US 2009/0163998 A1
(43) Pub. Date: Jun. 25, 2009

(54) ENDOPROSTHESIS HAVING RINGS LINKED BY FOOT EXTENSIONS

(75) Inventor: Brendan Casey, Galway (IE)

Correspondence Address: WORKMAN NYDEGGER, 1000 EAGLE GATE TOWER,, 60 EAST SOUTH TEMPLE, SALT LAKE CITY, UT, 84111, US

(73) Assignee: ABBOTT LABORATORIES VASCULAR ENTERPRISES LIMITED, Dublin (IE)

(21) Appl. No.: 11/961,775

(22) Filed: Dec. 20, 2007

(51) Int. Cl. A61F 2/82

(52) U.S. Cl. 623/1.16

(57) ABSTRACT

The present invention relates to an endoprosthesis having a plurality of web rings, pairs of which are coupled in one or more points by foot extensions protruding from at least one of the web rings. The endoprosthesis of the present invention provides a high level of flexibility and an elevated degree of scaffolding to a body lumen, and may be configured as a stent, graft, valve, occlusive device, trocar or aneurysm treatment device usable in a variety of intralumenal applications for treating vascular, coronary, biliary, esophageal, renal, urological and gastrointestinal conditions.

### FIELD OF THE INVENTION

The present invention relates to an endoprosthesis that is highly flexible and that provides elevated scaffolding properties to a vessel wall. More particularly, the present invention relates to an endoprosthesis that is formed by a plurality of web rings coupled in one or more points by foot extensions protruding from at least one of the rings.

### BACKGROUND OF THE INVENTION

Stents, grafts and a variety of other endoprostheses are known and used in interventional procedures, such as for treating aneurysms, lining or repairing vessel walls, filtering or controlling fluid flow, and expanding or scaffolding occluded or collapsed vessels. Such endoprostheses can be delivered and used in virtually any accessible body lumen of a human or animal and can be deployed using technologies known in the art.

More particularly, an endoprosthesis is typically delivered to a desired deployment site inside a body lumen by coupling the endoprosthesis to a catheter that is guided to the target location. To facilitate delivery, the endoprosthesis must have a flexible longitudinal profile to facilitate travel to the desired deployment site, which may be difficult to access by the treating physician through the tortuous pathway of the patient's anatomy. Therefore, it would be desirable to provide the endoprosthesis with a sufficient degree of longitudinal flexibility during delivery to allow advancement through the anatomy to the deployed site.

Once deployed, the endoprosthesis must satisfy a variety of performance characteristics. For example, the endoprosthesis should have sufficient rigidity or outer bias to perform its intended function, such as opening a lumen or supporting a vessel wall. Similarly, the endoprosthesis should be longitudinally flexible in its expanded state so that it will not kink, deform or fracture when deployed in a curved vessel. The endoprosthesis should provide an elevated and consistent degree of scaffolding of the vessel wall, and in certain applications such as the treatment of atherosclerosis in the carotid arteries, prevent plaque from protruding into the artery. Therefore, it would be desirable for the endoprosthesis to provide a substantially uniform or otherwise controlled scaffolding of the vessel wall while retaining a stable architecture during operation.

One type of endoprosthesis is the stent, which is used for the treatment of atherosclerotic stenosis in blood vessels. After a patient undergoes a percutaneous transluminal angioplasty or similar interventional procedure, a stent may be deployed at the treatment site to maintain patency of the vessel. The stent may include radio-opaque markers to facilitate delivery to the target location and may be loaded with a beneficial agent, acting as a delivery platform to reduce restenosis or the like.

Numerous endoprosthesis designs and constructions have been developed to address one or more of the performance characteristics summarized above. For example, the endoprosthesis may be formed by multiple rings connected longitudinally either at connection sections or with connector elements. The number of connection sections or connector elements and the thickness of the struts forming the rings control the flexibility of the endoprosthesis. A variety of stent designs are disclosed in U.S. Pat. No. 4,580,568 to Gianturco; U.S. Pat. No. 5,102,417 to Palmaz; U.S. Pat. No. 5,104,404 to Wolff; U.S. Pat. No. 5,133,732 to Wiktor; U.S. Pat. No. 5,292,331 to Boneau; U.S. Pat. No. 5,514,154 to Lau et al.; U.S. Pat. No. 5,569,295 to Lam; U.S. Pat. No. 5,707,386 to Schnepp-Pesch et al.; U.S. Pat. No. 5,733,303 to Israel et al.; U.S. Pat. No. 5,755,771 to Penn et al.; U.S. Pat. No. 5,776,161 to Globerman; U.S. Pat. No. 5,895,406 to Gray et al.; U.S. Pat. No. 6,033,434 to Borghi; U.S. Pat. No. 6,099,561 to Alt; U.S. Pat. No. 6,106,548 to Roubin et al.; U.S. Pat. No. 6,113,627 to Jang; U.S. Pat. No. 6,132,460 to Thompson; U.S. Pat. No. 6,331,189 to Wolinsky et al.; and U.S. Pat. No. 7,128,756 to Lowe et al., the entireties of which are incorporated herein by reference.

Some of these publications teach endoprosthesis, in which flexibility and stress absorption are increased by coupling adjacent web rings with connectors having different shapes (for example, rectilinear, “N” or “W” shapes). Unfortunately, such connectors cause the web rings to become spaced apart, decreasing the scaffolding properties of the endoprosthesis. Conversely, if the number of connectors between web rings is increased to improve scaffolding, the endoprosthesis becomes less flexible. Therefore, although the various endoprosthesis designs that have been developed to date may address one or more of the desired performance characteristics, there remains a need for a more versatile design for an endoprosthesis that allows improvement of one or more performance characteristics without sacrificing the remaining characteristics.

### SUMMARY OF THE INVENTION

The present invention relates to an endoprosthesis for delivery into a body lumen that has a plurality of web rings, pairs of which are coupled in one or more points by foot extensions protruding from one or both of the web rings. This endoprosthesis is highly flexible and provides an elevated degree of scaffolding to the body lumen, and may be configured as a stent, graft, valve, occlusive device, trocar or aneurysm treatment device for treating vascular, coronary, biliary, esophageal, renal, urological and gastrointestinal conditions.

In one embodiment, the endoprosthesis is a stent having a web structure that is expandable from a contracted configuration to an expanded configuration and that is formed by a plurality of longitudinally adjacent web rings. Each of the web rings includes web elements that that are disposed circumferentially around a longitudinal axis of the stent and are adjoined in sequence.

The web elements have a crown shape defined by a central member disposed essentially parallel to the longitudinal axis of the stent when the stent is in the contracted configuration and by end members that extend from the central member at obtuse angles. Pairs of the web elements are joined one to the other at junctions. In one embodiment of the invention, a junction in a first web ring is shaped as a foot extension and is coupled to a second junction in a second web ring that has an arcuate shape. The foot extension includes a sole portion that extends from one of the pair of web elements and a toe portion interposed between the sole portion and a second web element in the pair. The sole and the toe portions may each have an essentially arcuate shape, or the sole portion may be essentially rectilinear while the toe portion has an essentially arcuate shape. Further, the sole portion may be disposed essentially perpendicular to the longitudinal axis of the endoprosthesis or in a transversal direction.

In different embodiments of the invention, the first and the second web rings are connected by a plurality of foot extensions disposed circumferentially, and all or only some junctions in the first web ring may be shaped as foot extensions and be coupled to junctions in the second web ring. In one embodiment, the first and/or the second web rings include foot extensions that extend from junctions that are not coupled to other junctions in an adjacent web ring.

In different embodiments of the invention, the junctions in adjacent web rings that are coupled to one another may be longitudinally aligned, or may be laterally offset. Further, a foot extension may be coupled to a junction in an adjacent web ring at the sole portion, at the toe portion, or at segments connecting the sole portion to the toe portion.

The design of the foot extension may also include other portions in addition to the sole and toe portions. For example, the foot extension may include a heel portion interposed between the sole portion and one of the web elements. In different embodiments, the sole and the toe portions may have equal or different widths.

The central member and the first and second end members of the web elements may be essentially rectilinear, or have multi-segment or curved profiles, and the obtuse angles between the central member and the first and second member may be essentially equal or different. Further, the web elements in each web ring are nested one into the other in the contracted configuration, and in one embodiment the web elements of one web ring are oriented at approximately 180 degrees in relation to the web elements in a neighboring web ring.

The endoprosthesis of the present invention may be configured to self-expand from the contracted delivery configuration to the expanded deployed configuration, or may be expanded by applying a radial pressure to an interior surface of the endoprosthesis, for example, by inflating a balloon disposed within the endoprosthesis.

### BRIEF DESCRIPTION OF THE DRAWINGS

The drawings constitute a part of this specification and include exemplary embodiments of the invention, which may be embodied in various forms. It is to be understood that in some instances various aspects of the invention may be shown exaggerated or enlarged to facilitate an understanding of the invention.

FIG. 1 illustrates a schematic view of an endoprosthesis constructed according to the principles of the present invention.

FIG. 2 illustrates a detail view of the web structure of the endoprosthesis of FIG. 1 in a contracted condition.

FIG. 3 illustrates a detail view of the web structure of the endoprosthesis of FIG. 1 in an expanded condition.

FIG. 4 illustrates a foot extension according to a first embodiment of the invention.

FIG. 5 illustrates a connector having struts linked by foot extensions according to a second embodiment of the invention.

FIG. 6 illustrates a connector having struts linked by foot extensions according to a third embodiment of the invention.

### DETAILED DESCRIPTION OF EMBODIMENTS OF THE INVENTION

Detailed descriptions of embodiments of the invention are provided herein. It is to be understood, however, that the present invention may be embodied in various forms. Therefore, the specific details disclosed herein are not to be interpreted as limiting, but rather as a representative basis for teaching one skilled in the art how to use the present invention in virtually any detailed system, structure or manner.

The present invention relates to an endoprosthesis having a plurality of web rings, pairs of which are coupled in one or more points by foot extensions protruding from at least one of the web rings.

An endoprosthesis constructed according to the principles of the present invention is highly flexible and provides an elevated degree of scaffolding to a body lumen. Such endoprosthesis may be configured as a stent, graft, valve, occlusive device, trocar or aneurysm treatment device and may be used for a variety of intralumenal applications, including vascular, coronary, biliary, esophageal, renal, urological and gastrointestinal. For ease of description and without restrictive intent, the following embodiments of the present invention will be described hereinafter with reference to a stent.

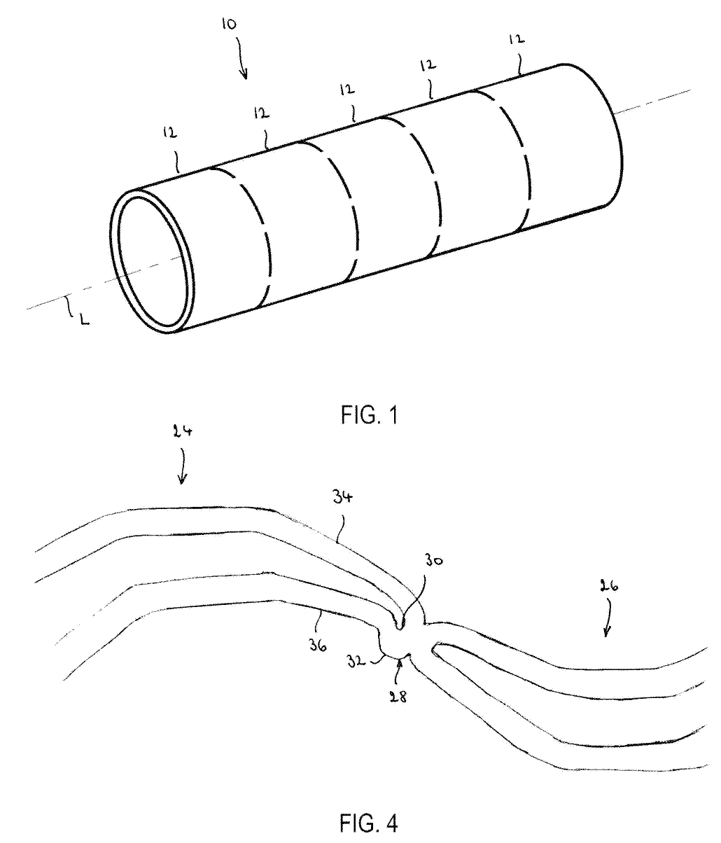

Turning first to FIG. 1, a first embodiment of the invention relates to a stent 10 having an essentially tubular body that is expandable from a contracted configuration, used for delivery of stent 10 to the target vessel, to an expanded configuration, used for scaffolding the target vessel after stent 10 has been delivered. The tubular body of stent 10 may have a variety of shapes, for example, may be cylindrical, frustoconical, or be shaped like a hyperboloid.

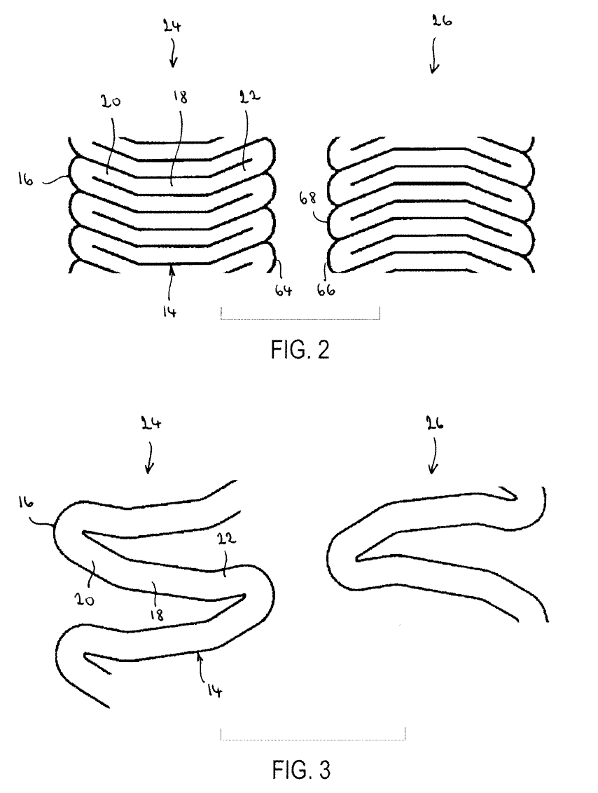

Stent 10 is formed by a web structure defined by a plurality of web rings 12 that are disposed one next to the other along a longitudinal axis L. As illustrated in FIGS. 2 and 3, each of web rings 12 is formed by a plurality of web elements 14 (shown in FIG. 2 in the contracted configuration and in FIG. 3 in the expanded configuration) that are disposed circumferentially around longitudinal axis L. Pairs of web elements 14 are joined one to the other by junctions 16, which are shaped like arcuate segments in the illustrated embodiment.

Each of web elements 14 includes a central member 18, which is situated in a direction essentially parallel to longitudinal axis L when stent 10 is in the contracted configuration of stent 10, and first and second end members 20 and 22 that extend from the terminal ends of central member 18 at obtuse angles. The angles between central member 18 and first member 20, and between central member 18 and second member 22 are preferably the same, but in different embodiments of the invention may be different one from the other.

Web elements 14 are nested one into the other in the contracted configuration, and the web elements in a first web ring 24 may be disposed in an opposite direction (that is, at 180 degrees) with respect to the web elements in a second web ring 26. Further, FIGS. 2 and 3 illustrate central member 18 and first and second end members 20 and 22 as having a rectilinear shape, but central member 18 and first and second end members 20 and 22 may have other shapes, for example, may have multi-segment shapes or curved shapes. Detailed descriptions of the web elements illustrated in FIGS. 2 and 3 can be found in U.S. Patent Application Publication Nos. 2004/0193250, and 2005/0004651, U.S. Pat. Nos. 6,682,554 and 6,602,285, International Patent Publication No. WO 00/13611, and German Patent Publication No. 19840645, the entireties of which are incorporated herein by reference.

Stent 10 may be manufactured from a variety of biocompatible materials known in the art, including plastic and metal materials, and may be deployed at the target vessel using techniques also known in the art, either by inflating a balloon coupled to the catheter, or if stent 10 is manufactured from a shape memory material such as Nitinol (a nickel-titanium alloy), by causing stent 10 to self-expand until contact with the vessel wall is established. Stent 10 may also be coated with a therapeutic material, for example, a restenosis-inhibiting material or an immunosuppressant such as everolimus.

Turning now to FIG. 4, at least some of the junctions in first web ring 24 or/and in second web ring 26 may be shaped as foot extensions 28 and may serve as the points where first web ring 24 is coupled to second web ring 26. More particularly, foot extension 28 includes a first portion that extends from a first web element 34 in first web ring 24 and that provides sole portion 30 of foot extension 28, and a second portion that provides toe portion 32 of foot extension 28 and is interposed between sole portion 30 and a second web element 36.

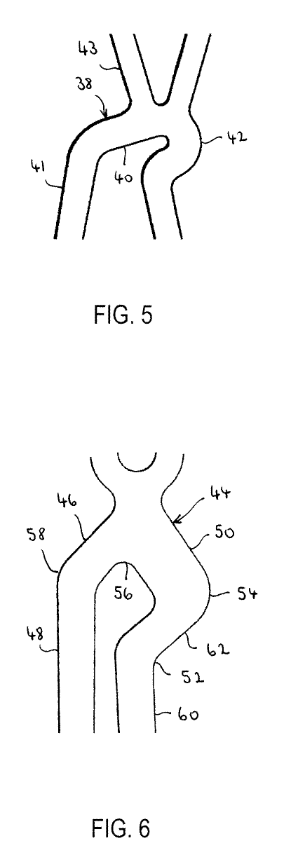

In the embodiment illustrated in FIG. 4, both sole portion 30 and toe portion 32 have arcuate shapes. In different embodiments of the invention, the foot extension may have different configurations. For example, FIG. 5 illustrates a foot extension 38 that includes a sole portion 40 that is essentially rectilinear in shape and a toe portion 42 that is arcuate. In this embodiment, sole portion 38 may be disposed in a circumferential direction perpendicularly to longitudinal axis L or, as shown in FIG. 5, in a direction that is not perpendicular to longitudinal axis L. In addition, all foot extensions 40 connecting first web ring 41 to second web ring 43 may be oriented in the same direction (for example, with further reference to FIG. 5, all toe portions 42 may be directed towards the left side of FIG. 5 or all toe portions 42 may be directed towards the right side of FIG. 5) or may be oriented in varying directions (for example, still with reference to FIG. 5, some toe portions may be directed towards the left side of FIG. 5 and some towards the right side).

A foot extension as described herein provides for increased flexibility in comparison with stent designs where no foot extension is present, while at the same time providing for elevated scaffolding properties. For example, with reference to foot extension 28 in FIG. 4, the arcuate portions of foot extension 28 (namely, sole portion 30, toe portion 32, and the curved segments connecting sole portion 30 to toe portion 32 and to first and second web elements 34 and 36) provide for an increased flexibility of stent 10 both by providing areas of flexure that can absorb tension, compression, flexure or torsion stresses applied to stent 10 and also provide stent 10 with an increased ability to expand or contract longitudinally, reducing foreshortening during stent expansion. In addition, foot extension 28 increases the surface density of stent 10, therefore, provides increased scaffolding properties to stent 10 in comparison with stents of similar construction, in which foot extensions 28 are not present.

Referring now to FIG. 6, an alternative embodiment of the invention relates to an endoprosthesis having a foot extension 44 that further includes a heel portion 46 defined by a segment (either rectilinear or arcuate) interposed between first web element 48 and sole portion 50. In this embodiment as well as in the preceding embodiments, all flexure areas 52, 54, 56 and 58 that connect the various portions of foot extension 44 one to the other are preferably rounded, in order to minimize stress concentration and reduce the risk of fracture under stress. Also as shown in FIG. 6, but as possible in each of the preceding embodiments, the various portions of foot extension 44 may have different widths, and may also have widths different form the widths of first web element 48 and/or second web element 60. For example, in the embodiment depicted in FIG. 6, sole portion 50 is wider than heel portion 46 and toe portion 62.

The foot extensions described above are only representative of the numerous, other possible designs of a foot extension. Other foot extension designs are disclosed in U.S. Pat. No. 7,128,756 to Lowe et al. and in U.S. Patent Application Publication Nos. 2005/0107865 to Clifford et al., 2006/0015173 to Clifford et al., 2006/0142844 to Lowe et al., 2007/0021834 to Young et al., and 2007/0021827 to Lowe et al., the entireties of which are incorporated herein by reference.

While FIGS. 4 and 5 show that the first and the second web rings may be adjoined at the sole portion of the foot extensions, in other embodiments of the invention the first and the second web ring may be adjoined at other portions of the foot extension. For example, FIG. 6 shows that the two neighboring web rings may be coupled at flexure area 56 between heel portion 46 and sole portion 50, but a person skilled in the art will appreciate that two neighboring web rings may be coupled in still different points of a foot extension.

In different embodiments, each junction in a web ring may be coupled to another junction in an adjacent neighboring web ring, or only one every number of junctions in a web ring may be coupled to a junction in an adjacent web ring. Further, while the foot extensions in the preceding embodiments have been described only as coupling junctions in two neighboring web rings, other foot extensions may protrude from junctions and not be used for coupling neighboring web rings, but only for increasing the flexibility of the web structure. In a further different embodiment, two foot extensions may be coupled to each other, thereby connecting neighboring web rings via two foot extensions. In this embodiment the sole portion of the foot extension of the first web ring may be adjoined to the sole portion of the foot extension of the second web ring or the tip portion of the foot extension of the first web ring may be adjoined to the tip portion of the foot extension of the second web ring. However, it will be appreciated by a person skilled in the art that two neighboring web rings may be coupled in still different points of the foot extensions.

The foot extensions described hereinbefore may couple junction between web elements that are longitudinally aligned, such as junctions 64 and 66 in FIG. 2, or junctions that are laterally offset, for example, junctions 64 and 68 in FIG. 2. Coupling junctions that are laterally offset reduces the relative rotations of neighboring web rings when bending, torsion, tension, compression or expansion forces are applied to stent 10, because the foot extensions tend to absorb some or all of the rotational forces acting between adjacent web rings when such rotational forces become applied.

Based on the foregoing, it can be seen that an endoprosthesis having a web structure that includes foot extensions as described above, provides elevated scaffolding properties and also an elevated degree of flexibility. While the invention has been described in connection with the above described embodiments, it is not intended to limit the scope of the invention to the particular forms set forth, but on the contrary, it is intended to cover such alternatives, modifications, and equivalents as may be included within the scope of the invention. Further, the scope of the present invention fully encompasses other embodiments that may become obvious to those skilled in the art and the scope of the present invention is limited only by the appended claims.

What is claimed is:

1. An endoprosthesis for delivery in a body lumen comprising:
a web structure defining an essentially tubular body expandable from a contracted configuration to an expanded configuration;
a plurality of longitudinally adjacent web rings defining the web structure; and
a plurality of sequentially adjoined web elements defining the web rings, the web elements being disposed circumferentially around a longitudinal axis of the essentially tubular body, each of the web elements comprising a central member having a first and a second ends, the central member being disposed essentially parallel to the longitudinal axis in the contracted configuration, a first end member being connected to the first end at a first obtuse angle and a second end member being connected to the second end at a second obtuse angle, pairs of the web elements being sequentially adjoined at junctions,
wherein a first junction in a first web ring is shaped as a foot extension and is coupled to a second junction in a second web ring,
wherein the foot extension comprises a first portion extending from a first web element in the first web ring, the first portion providing a sole portion of the foot extension, and
wherein the foot extension comprises a second portion interposed between the sole portion and a second web element in the first web ring, the second portion providing a toe portion of the foot extension.

2. The endoprosthesis of claim 1, wherein the sole portion and the toe portion each have an essentially arcuate shape.

3. The endoprosthesis of claim 1, wherein the sole portion has an essentially rectilinear shape and the toe portion has an essentially arcuate shape.

4. The endoprosthesis of claim 3, wherein the sole portion is disposed essentially perpendicular to the longitudinal axis.

5. The endoprosthesis of claim 1, wherein the first and the second web rings are connected by a plurality of foot extensions disposed circumferentially.

6. The endoprosthesis of claim 1, wherein the first and/or the second web rings comprise foot extensions extending from junctions not coupled to other junctions.

7. The endoprosthesis of claim 1, wherein the first and the second junction are longitudinally aligned.

8. The endoprosthesis of claim 1, wherein the first and the second junctions are laterally offset one from the other.

9. The endoprosthesis of claim 1, wherein the foot extension is coupled to the second junction at the sole portion, at the toe portion, or at a segment between the sole and toe portion.

10. The endoprosthesis of claim 1, wherein the foot extension further comprises a third portion interposed between the sole portion and the first web element, the third portion providing a heel portion of the foot extension.

11. The endoprosthesis of claim 1, wherein the endoprosthesis is a stent.

12. The endoprosthesis of claim 1, wherein the central member and the first and the second end members are essentially rectilinear.

13. The endoprosthesis of claim 1, wherein the central member and the first and the second end members have multi-segment or curved profiles.

14. The endoprosthesis of claim 1, wherein the first and the second obtuse angles are essentially equal.

15. The endoprosthesis of claim 1, wherein the sole portion and the toe portion are segments of equal width.

16. The endoprosthesis of claim 1, wherein the web elements of each web ring are nested one into the other in the contracted configuration.

17. The endoprosthesis of claim 16, wherein the web elements in the first web ring are oriented at approximately 180 degrees in relation to the web elements in the second web ring.

18. The endoprosthesis of claim 1, wherein the web structure is configured to self-expand from the contracted configuration to the expanded configuration.

19. The endoprosthesis of claim 1, wherein the web structure is configured to expand from the contracted configuration to the expanded configuration by application of a radial pressure to an interior surface of the essentially tubular body.
